(19) United States
(12) Patent Application Publication
(10) Pub. No.: US 2013/0204115 A1
(43) Pub. Date: Aug. 8, 2013

(54) COMPUTER BASED ANALYSIS OF MRI IMAGES

(71) Applicants: Erik B. Dam, Copenhagen (DK); Rabia L. Granlund, Copenhagen (DK); Martin Lillholm, Niva (DK)

(72) Inventors: Erik B. Dam, Copenhagen (DK); Rabia L. Granlund, Copenhagen (DK); Martin Lillholm, Niva (DK)

(73) Assignee: Synarc Inc., Newark, CA (US)

(21) Appl. No.: 13/701,102

(22) PCT Filed: May 25, 2011
(86) PCT No.: PCT/EP2011/058588

§ 371 Date: Apr. 5, 2013

(30) Foreign Application Priority Data
Jun. 1, 2010 (GB) 1009101.5

(51) Int. Cl. A61B 5/00; A61B 5/055

(52) U.S. Cl. CPC A61B 5/4509; A61B 5/055; A61B 5/7282; A61B 5/7275; A61B 5/4528; A61B 5/7225; A61B 5/4514
USPC 600/410

(57) ABSTRACT

A method for computer based analysis of a low field MR image including a trabecular region of bone for extracting from said image diagnostic information by applying a trained statistical classifier which has been trained on similar labelled according to the severity of a trabecular bone altering disease suffered at the time or later. For each image in the training set a region of interest (ROI) is defined, textural information relating to the intensities of voxels within the ROI is obtained, and combinations of features of said textural information are found which suitably classify the images according to said labelling. An image under study is treated similarly and features of said textural information for the voxels within the ROI of the image are combined as learnt in the training of the classifier to estimate a level of said trabecular bone altering disease or propensity to develop said bone altering disease or a level thereof associated with said image.

The present invention relates to the computer based analysis of low field MRI images showing a trabecular region of bone in order to extract therefrom biomarker information relating to a trabecular bone altering disease condition.

It is known that various conditions including osteoarthritis, rheumatoid arthritis and osteoporosis produce changes in the structure of trabecular bone which can be seen in high resolution MRI images (U.S. Pat. No. 7,643,664; KR20090042645). US2002/0015517 acquired images at 1.5 T and used sub-voxel processing to enhance the apparent resolution to quantify trabecular architecture for predicting fracture risk in osteoporosis. However, even then no evidence was presented that such risk was successfully predicted.

Where low field MRI has been applied to attempt to characterise trabecular bone, it has been on the basis of measuring gross properties of a part of a bone, such as the proton density within a heel to quantify its trabecular bone content (US2002/0022779—using 0.21 T) rather than by image analysis.

WO2007/062255 describes analysis of Fourier representation of MR signals, which have not been converted into an image. The disclosure is vague and there is no evidence presented that it provides information of diagnostic significance.

The present invention is based on the discovery that when low resolution MRI images are appropriately analysed by computer based statistical methods, diagnostic information of value can be extracted even though the individual trabeculae are not resolved and it remains undemonstrated that a radiologist would be able to extract diagnostic information by eye.

Whilst the invention is applicable to other trabecular bone altering diseases, we shall illustrate the relevant concepts with reference to osteoarthritis (OA).

OA is a widespread disease which affects up to 80% of the population over 65 years [1]. The disease is a degenerative joint disease causing pain and decreased mobility leading to an impaired quality of life. Currently only treatment of the symptoms of OA is documented.

Developing biomarkers to diagnose accurately and monitor disease progression is a key element in the evaluation of the efficacy of new treatments. This is far from trivial because especially in the initial stages of OA the changes are subtle.

The pathogenesis of OA is a complex chain of events in the whole joint but until recently the main focus has been on the cartilage. The low friction of cartilage allows for effortless joint movement. Furthermore, cartilage works closely together with the subchondral bone, including the trabecular bone, adapting to and mediating mechanical stress.

Cartilage loss and overall bone remodelling are central in the progression of OA, and have been studied in many ways. The gold standard method for measuring OA progression in medical images is currently the Kellgren & Lawrence (KL) score [2] determined from radiographs. The KL score measures the joint space narrowing and thus indirectly cartilage degradation together with other OA features as osteophyte formation, sclerosis, and deformity of bone contour [3]. The bone structure has been investigated mainly from radio-graphs, micro computed tomography (μCT), or high-resolution magnetic resonance imaging (MRI). In a radiograph only bone and other hard tissues are visible. Furthermore, radiographs are two-dimensional and thus most likely to have imaging artefacts. μCT is only applicable ex vivo, so high field MRI is used increasingly in OA research. It is well suited for imaging other tissues than cartilage in the joint such as the trabecular bone structure and its changes [3]. High field MR images give generally a more detailed view than radiographs and OA can be detected much earlier. Fully automatic methods exist for quantifying and qualifying cartilage [4], [5]. No fully automatic quantitative method exists for monitoring trabecular changes in MRI.

### A. Magnetic Resonance Imaging

MR scanners exist using various field strengths and providing various resolutions. The magnetic fields generally range from 0.1 T to 3.0 T. When the field increases the signal-to-noise ratio improves and the potential resolution increases. With a high resolution MR scanner the trabecular structure of the bone is directly visible. It is therefore possible directly to monitor and determine its importance in development and progression of OA [3]. However, these high resolution MR scanners are extremely costly and the scanning time increases with the resolution.

Lower field MRI are similarly lower in resolution but also in cost which make them attractive for clinical studies. Apart from the general whole-body scanners, low field scanners are also found in smaller versions designed to scan specific body parts such as the extremities. With such a scanner the magnetic coil is placed very close to the structure to be imaged which increases the field homogeneity, as compared to the whole-body scanner [3].

Low field scanners may be considered to include particularly those which use a field not in excess of 0.5 T. Generally the field will be at least 0.1 T.

A thorough review of semiquantitative and quantitative measurement methods in OA was presented by Eckstein et al. [6]. Current semiquantitative measures of whole joints cover various structures within the knee joint. An example of a semiquantitative method is the WORMS (Whole-organ MR imaging score) [7] which has been used in several clinical trials and epidemiologic studies [8]. Another whole joint semiquantitative method is the Knee Osterarthritis Scoring System (KOSS) [9]. Both methods analyze OA features such as cartilage, bone marrow lesions, subchondral cysts, meniscus, and osteophytes. The features are scored by a trained radiologist resulting in an overall score of the knee. Neither of the covered structures concern the trabecular network of plates and rods.

Until now bone quality and bone strength have been measured by Bone Mineral Density (BMD) or bone histomorphometry measures such as trabecular thickness and trabecular number [10]. BMD obtained from DEXA scans is often used in osteoporosis as a measure of the strength of the bone. As the name indicates BMD is a measure of the bone density which is not necessarily closely related to the quality and strength of the trabecular bone. It has been suggested that BMD is insufficient when analyzing the trabecular bone in relation to OA [11].

A way of semi-automatically quantifying the trabecular structure and to monitor changes occurring with OA is the Fractal Signature Analysis (FSA) from macroradiography [12], [13]. A region of interest (ROI) within the subchondral tibial trabecular bone was analyzed by FSA, measuring the fractal dimension of the trabeculae and quantifying the variation of the fractal dimension with the size of the structures. The results showed that FSA can quantify changes in the trabecular bone in knee OA patients. This methodology however requires high resolution images.

A semi-automatic bone structure method based on histomorphometry was presented in [14]. From micro computed tomography scans (μCT) the femoral and tibial bone was investigated ex vivo. The scans were binarized and from the resulting trabecular structure the trabecular number, thickness, etc. was computed together with measures of how rod-like or plate-like the bone appeared. The results showed that the trabecular structure differed between the healthy and the arthritic knee. Furthermore, Patel et al. showed trabecular variations in relation to the depth in the bone, between tibia and femur, and between the medial and lateral compartments.

A fully automatic quantitative method for segmentation of cartilage is presented in [5]. Dam et al. show that it is possible to extract cartilage imaging markers from low-field MRI to separate healthy and OA knees. Furthermore, prediction of cartilage loss based on imaging markers was also demonstrated [4]. Among the fully automatic MRI markers derived from cartilage are volume, thickness, surface curvature, and homogeneity. As previously mentioned some of these markers are capable of discriminating between healthy and OA knees both in the early stages and the later stages of OA [5], [15].

The present invention provides in a first aspect a method for computer based analysis of a low field MR image including a trabecular region of bone for extracting from said image diagnostic information, said method comprising applying to said image a trained statistical classifier which has been trained on a training set of low field MRI images containing said trabecular region of bone each of which images has been labelled according to the severity of a trabecular bone altering disease suffered by a person from whom the image derived, wherein in said training of the classifier, for each image in the training set a region of interest (ROI) was defined, and textural information relating to the intensities of voxels within the ROI was obtained, and combinations of features of said textural information were found which suitably classify said training set images according to said labelling, and wherein, in applying said trained statistical classifier to said image, in a computer the region of interest (ROI) is found in said image, and textural information relating to the intensities of voxels within the ROI of the kind used in training the classifier is obtained, and features of said textural information for the voxels within the ROI of the image are combined as learnt in the training of the classifier to estimate a level of said trabecular bone altering disease or propensity to develop said bone altering disease or a level thereof associated with said image.

If the training set images are selected to show (a) healthy bone and (b) bone of a patient suffering from a trabecular bone altering condition, then the analysis of the image under test may provide a categorisation of that image as showing some present degree of disease, which may be regarded as a diagnostic biomarker.

Of course, it may be useful to repeat the taking of the image to be analysed after some period and to re-run the analysis and to compare the values of such a diagnostic biomarker obtained originally and on at least one such future occasion. This may reveal progress or lack of it in the development or cure of the disease and may be associated with intervening events such as the use of a therapy. This may enable the marker to be used as an efficacy biomarker in the study of such a treatment.

However, the training set images may be selected such that they all appear either to the eye or to machine analysis as per the invention to relate to presently healthy bone, but they may be categorised as coming from (a) patients who remain healthy through some extended period of a longitudinal study and (b) patients who develop disease in such a study. Thus, although the images are labelled according to the severity of disease of a patient from whom it originates, it is not necessarily the case that the disease is or was manifest at the time the image was taken. In such a case, the result of the analysis may be to categorise the image under test as coming from an apparently presently healthy person who is more or less likely to develop disease within a certain period, i.e. the result may be viewed as a prognostic biomarker.

Thus, optionally, said training set images may have been labelled according to the severity of trabecular bone altering disease suffered by the person to whom the image relates at the time of taking the image.

Alternatively, said training set images may have been labelled according to the severity of trabecular bone altering disease suffered by the person to whom the image relates at a time subsequent to the time of taking the image and wherein the image was taken at a time when the said person did not suffer from the disease or suffered from the disease to a lesser extent.

The present or future disease state of the person from whom the image comes may be regarded as an example of metadata relating to the image, i.e. it is information relevant to the condition of the bone shown in the image which is not derived from the image itself. Other forms of metadata may additionally be used in labelling the images, e.g. the nature of therapy to which the person has been exposed, whether they belong to a group of responders or non-responders to some therapy, depending on clinical symptoms such as pain, and so on.

Said trabecular bone altering disease may be arthritis (including osteoarthritis and rheumatoid arthritis), Paget's disease, or osteoporosis. Additionally, the methods are also appropriate for investigating potential bone altering effects from systemic or metabolic diseases (such as hyperparathyroidism, hyper- and hypothyroidism) or from therapy (such as treatments like bisphosphonates, vitamin D, hormones, selective estrogen receptor modulator, prednisolone, anabolic androgens, or parathyroid hormone).

The training set images and the image to be analysed are preferably acquired using an MRI apparatus having a field strength of not more than 0.5 T. Generally the field strength will be not less than 0.1 T, e.g. from 0.125 T to 0.225 T.

Said textural information may include textural information obtained by applying to the image one or more of the following filters:

N-jet, Structure Tensor, Hessian, Gradient, Third order derivatives, and Gradient Magnitude
and deriving for each filtered image one or more of the mean, standard deviation and Shannon entropy.

Preferably, said filters are applied at multiple (e.g. 3) scales.

Said textural information may further include textural information obtained by deriving from the unfiltered image one or more of the mean, standard deviation and Shannon entropy.

Preferably, said textural information includes textural information obtained by applying to the image at least the N-jet, Structure Tensor and Hessian filters at multiple scales and deriving for each filtered image one or more of the mean, standard deviation and Shannon entropy.

Said estimation is preferably combined with one or more other biomarkers estimating the present or future extent of said trabecular bone altering disease in the person from whom the image derives, so as to form a composite biomarker.

Examples of such other biomarkers include a biochemical cartilage breakdown product measure (especially in the case of arthritis), a biochemical bone breakdown product measure (especially in the case of arthritis or osteoporosis), cartilage volume, cartilage thickness, cartilage smoothness, cartilage curvature, and cartilage homogeneity.

The invention includes a method for the development of a statistical classifier for computerised classification of a low field MR image including a trabecular region of bone for extracting from said image diagnostic information, said method comprising training a statistical classifier on a training set of low field MR images containing said trabecular region of bone each of which images has been labelled according to the severity of a trabecular bone altering disease suffered by a person from whom the image derived, wherein in said training of the classifier, for each image in the training set a region of interest (ROI) is defined, and textural information relating to the intensities of voxels within the ROI are obtained, and combinations of features of said textural information are found which suitably classify said training set images according to said labelling.

Generally preferred features of the first aspect of the invention may be employed here too.

The invention will be further described and illustrated with reference to the accompanying drawings in which:

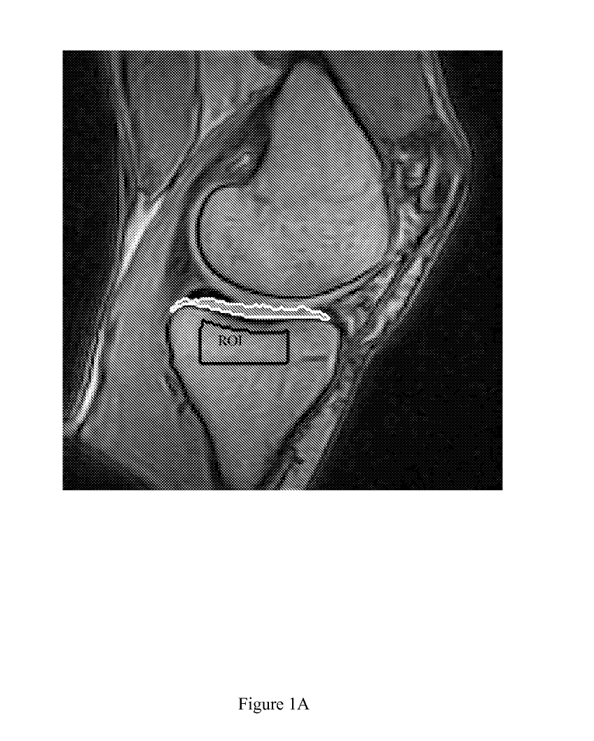

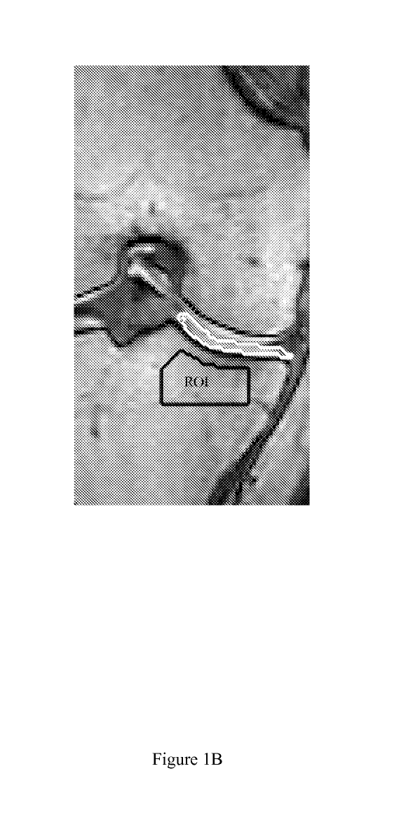

FIG. 1A shows a sagittal view of a knee. The dark outlined area marked ‘ROI’ is the automatically extracted ROI and the light outlined area is the given cartilage segmentation.

FIG. 1B. Shows a coronal view of a knee. The given cartilage segmentation and the ROI are marked as in FIG. 1A.

FIG. 1C. Shows an axial view of a knee. The automatically extracted ROI is again marked.

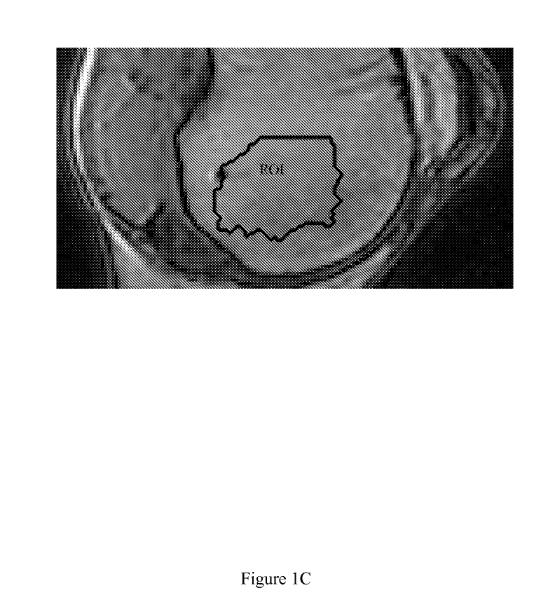

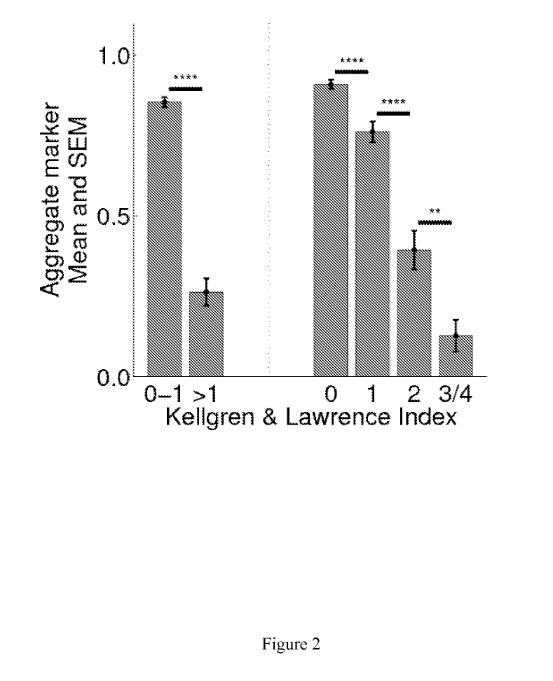

FIG. 2 shows the probability of images relating to a healthy knee as obtained in the investigation reported below.

In an illustrative embodiment of the invention, MR images were acquired as follows. The MRI scanner was an Esaote C-Span low-field 0.18 T scanner dedicated for imaging of the extremities. The scanner parameters were as follows: Turbo 3D T1 sequence, 40° flip angle, 50 ms repetition time, and 16 ms echo time. While scanning, test subjects were in a supine position with no load-bearing. Acquisition time was approximately 10 minutes.

The size of the scans is 70×170×170 voxels after automatically removing boundaries containing no information. Spatial in-plane resolution was 0.70×0.70 mm² with a slice thickness ranging between 0.7 and 0.94 mm depending on joint size. The most common distance was 0.78 which made the voxels nearly isotropic.

The data set thus acquired consisted of 3D MR scans from 159 test subjects. After exclusion of scans due to acquisition errors, 311 knee scans were included. Examples of scan slices are seen in FIGS. 1, 2, and 3. The population characteristics were: age 56±16, BMI 26±4, 47% female, and 19% with radiographic OA (KL>1).

All scans have been scored by a gold standard method for measuring knee OA: the Kellgren & Lawrence score [2] determined by a radiologist from radiographs.

The scores ranged from 0 to 4, where KL0 indicates a healthy knee, KL1 borderline, and KL2-4 defines a knee with moderate to severe OA. The distribution of knees in the data set is shown in Table I.

TABLE I

| KL score | Number of knees |
|---|---|
| 0 | 157 |
| 1 | 94 |
| 2 | 31 |
| 3 | 28 |
| 4 | 1 |
| Total | 311 |

When dividing in healthy/borderline and OA knees the percentage of knees in each group is 81% and 19% which makes the data set unbalanced.

The scans show the knee consisting of the femoro-tibial joint, which links the tibia and femur bone. The cortical shell appears as an almost black line around the bright trabecular bone within. The cartilage is shown as a bright layer on the surface of the articular bone.

The segmentation of cartilage was done in accordance with Folkesson et al. [16]. First, a fully automatic voxel classification including feature selection was performed by a kNN classifier. The classifier was trained on manual segmentations from 25 knee scans. The classification resulted in a binary segmentation of the tibial cartilage. Last, a statistical shape model made as described in Dam et al. [17] was deformed into the binary segmentation and this shape model formed the basis of bone structure analysis.

We shall now describe the following four steps: First region of interest (ROI) extraction is performed. Second the extraction of features within the region of interest is presented. Last, classification and selection of the best texture features is described.

The goal of ROI extraction is to choose automatically the ROI so that it lies within the trabecular bone and thus neither covers cartilage nor cortical bone. The ROI parameters were chosen by visual inspection of knee scans with varying degrees of OA, ensuring only trabecular structure within in the ROI. The resulting ROI was located from 2 to 13 mm below the cartilage, from 20 to 75% of the anterior/posterior cartilage, and from 20 to 60% of the medial/lateral cartilage, in average. An example of an automated ROI extraction is seen in FIGS. 1A, B and C.

To enhance the relevant information in the scans with respect to the progression of OA, feature extraction is performed to assist the classifier in determining if a scan is healthy or diseased. Because of the lack of knowledge of the underlying structures we are looking for, numerous generic features are extracted from the images. Therefore feature selection is subsequently performed to choose the best features.

A generic set of features which have proven to give good results for many patterns is the N-jet [24]. The N-jet filters result in a set of Gaussian derivative kernels up to order N. Here, 3-jet filters are included resulting in the Gaussian derivative kernels being up to and including the third order. Furthermore, non-linear combinations of the Gaussian derivative kernels were included such as the Structure Tensor [25], Hessian, and Gradient Magnitude. Last, the intensity image (that is to say the original unfiltered image) was added to the feature set. The above mentioned filters included features invariant to scale, rotation, and intensity so the features were expected to include the bone structures visible in the images. To keep the computational cost low only linear features and their non-linear combinations were extracted.

We aimed to analyze the bone structure at several scales to avoid missing important structures because of inappropriate scales. A small scale is included to encompass the trabecular structure. Furthermore, larger structures such as bone marrow lesions (BML) are possibly also changing with disease and thus the need for larger scale features. So, the scales are chosen as 1, 2, and 4 mm. Even though BMLs can be 1-2 centimeter in size and the largest scale is less than a centimeter BMLs are still expected to appear because the features are calculated across a region resulting in a summary effect.

Each feature extraction resulted in a feature image of the same size as the scan. For many of the filters the result of the extraction was a vector of three values, e.g. the Eigen vector of the Structure Tensor at some scale. The features were all treated separately so the Eigen vector became three individual features. As stated previously, the goal was to classify a knee scan as healthy or diseased. This raised the need for a single score of the whole knee for each feature image. This can be done in numerous ways. In [20] the intensity histogram within the ROI was used for classification while pixel classification followed by a fusion of classification probabilities into a single score was performed in [21]. Such methods would be applicable also in the practice of this invention. Here however, a classification of voxels was not necessary, and thus was not conducted. Furthermore, we wanted to keep the single scores simple. So, evident measures of the variation within the ROI: standard deviation (std), and Shannon entropy were computed for each feature image. Due to the summary effect of BMLs the mean of the ROI was computed in addition, resulting in three different feature measures.

When extracting the above mentioned features at three different scales and calculating the 3 feature measures for each feature image the total number of features for each image was 534.

The trained classifier may be of numerous kinds, however some may be better suited to the task than others. We wish to separate healthy and OA knees by a supervised classifier where the ground truth is the KL score. The performance of six different classifiers was evaluated with respect to their ability to classify the knee scans as healthy or osteoarthritic. Diverse classifiers were chosen because it was unknown how complicated the separation of the classes was and which type of classifier would perform best for this problem. A simple linear classifier, the Linear Discriminant Analysis (LDA), the corresponding quadratic classifier (QDA), the Nearest-Neighbor (NN), k-Nearest-Neighbor (kNN), the weighted Nearest-Neighbor (wNN) [19], and the weighted k-Nearest-Neighbor (wkNN) methods were tried. For the NN and kNN variants the Approximate Nearest Neighbor implementation was used [20].

Because the data is unbalanced with respect to the number of healthy and OA knees, cost functions such as the accuracy measure would be an inappropriate choice for this particular data set while the area under the ROC curve (AUC) is suitable. The AUC was therefore chosen as the measure comparing different feature sets.

Feature selection was performed to provide an appropriate feature set to the classifier to separate the healthy from the OA knees.

The best feature set was selected by Sequential Floating Forward Selection (SFFS) [21], [22]. SFFS is a classifier-dependent suboptimal method previously shown to have a performance comparable to the optimal methods [23], [24]. The algorithm initiates with the empty feature set. The first step is to iteratively include features to the feature set. Following every inclusion is the exclusion step where SFFS excludes a feature if the new feature set performs better than the previous feature set of the same size. Because of the floating nature of the method the ability to correct “mistakenly” added or removed features is a possibility. Therefore, the nesting problems of other suboptimal feature selection methods such as SFS [22] were avoided.

The features were weighted so a feature can be added repeatedly which results in an increasing weight of the feature. To ensure a manageable computation time a maximum number of features was set. When the maximum number of features was reached the algorithm stopped. The overall best feature set was chosen as the set with the highest performance regardless of its size. Preliminary experiments showed that already at a feature set size of 10 the training AUC did not improve significantly. The maximum number of features was therefore set to 20 which makes room for the algorithm to float backward and forward before reaching the maximum number of features.

The overall system for diagnosis of OA based on the trabecular bone structure of the tibia is summarized in the following computer executed algorithm:
1: for all knee scans do
2: Calculate ROI
3: Extract features
4: end for
5: for each evaluation do
6: Divide in data in training, validation, and test set
7: Normalize features for each set
8: Do SFFS until reaching maximum features
9: return Training AUC for feature sets of size 1 to max feat
10: Choose bone structure marker as the feature set with highest training AUC
11: Do classification by bone structure marker for test data
12: Calculate generalization AUC
13: end for
14: Calculate median results for all evaluations

When evaluating the performance of a given feature set for a given classifier two types of performance measures are considered: The training AUC and the generalization AUC. The training AUC generally describes how well the chosen features explain the training data while the generalization AUC describes how well the found features separate new data [25]. The goal is to maximize both performance measures by choosing the features that both entail a high training AUC and a high generalization AUC. The training AUC will normally always be larger than the generalization AUC because some degree of overfitting is inevitable. To avoid extensive overfitting of the feature set the goal is to minimize the difference between the training and the generalization AUC. Overfitting is particularly present when the number of features is large and the number of training samples is limited, like the data used here.

Designing the feature selection evaluation so that the chosen feature set generalizes well is important. The simplest way is to divide the data into three sets: training, validation, and test set [22, chapter 2]. The training set forms the training data for the classifier while the validation set is used for testing the performance of a given feature subset. Finally, when the optimal feature subset is found the generalization of the feature set is tested by classification of the test set.

To encompass the diversity of the data, cross-validation (CV) is widely used. By cross-validation the data is divided into the three above mentioned sets N times and hence N evaluations are performed. The performance is calculated as the median AUC over all N evaluations.

A special case of CV is Leave-one-out (LOO) where N equals the number of samples and the validation set consist of only one sample. The LOO scheme is expected to generalise better because the number of training samples is increased but it is more computationally expensive than CV.

Feature selection experiments compared the performance of the following six classifiers when doing feature selection: NN, wNN, kNN, wkNN, LDA, and QDA.

For each classifier cross-validation (CV) and Leave-one-out (LOO) were done. I was expected that LOO would perform better than CV when as here limited data is available and the feature space is high dimensional. For each, a total of 100 evaluations were done where the data set was randomly divided into subsets.

For CV the data was divided into three sets where ⅓ was used for training, ⅓ for validation, and ⅓ for test. When performing feature selection, this resulted in 104 training scans and 104 validation scans. Calculating the CV generalization, the training and validation sets were both included as training resulting in 208 training scans and 103 test scans.

In LOO the data was divided into two sets where ⅔ was used for training/validation and ⅓ for test. LOO feature selection included 208 scans, of which 1 was iteratively chosen for validation and 207 for training. The generalization of LOO was calculated on the test set of 103 scans, by training on all but 1 scan, resulting in 310 training scans. For both schemes, the overall training and test performance was calculated as the median of the 100 AUCs.

Each feature was normalized to zero mean and unit variance based on the distribution of the training set. Feature selection was performed with a maximum of 20 features. Determining k for the kNN classifier was determined by using the rule of thumb: k=√n, where n is the total number of training samples [28]. k is rounded to the nearest integer. For CV: k=10, LOO: k=14. For both NN and kNN, ANN eps=0 [27].

Previous research show that biochemical markers in combination with imaging markers of OA can result in good aggregate markers which improve both diagnosis and prognosis of knee OA [26]. Therefore, the performance of the developed bone structure marker was evaluated with respect to other types of biomarkers related to OA progression. The performance of the individual biomarkers was analyzed and compared to the developed bone structure marker. Furthermore, an aggregate marker was evaluated. To encompass different biomarkers both a biochemical marker and MRI markers were included. All presented OA biomarkers were previously evaluated in [26].

The biochemical marker is the urinary levels of collagen type II C-telopeptide fragments (CTX-II). CTX-II is an indicator of cartilage degradation. It was measured for each patient resulting in identical CTX-II values for the left and right knees, respectively, of the patient.

Five different MRI cartilage markers were included: volume, thickness, smoothness, curvature, and homogeneity. All markers were computed based on the cartilage segmentation described above. The volume marker describes the quantity of cartilage normalized to the joint size [16]. Thickness is measured as the mean thickness of the cartilage sheet [17]. The smoothness relates to the fine-scale surface curvature while the curvature marker measures the global bending of the cartilage sheet from [4]. The homogeneity was quantified as 1 minus entropy in the medial tibial compartment as in Qazi et al. [27] measuring the uniformity of the cartilage. All MRI cartilage markers were extracted from the same images used in this example.

The biomarkers were evaluated by the LDA LOO classification scheme described above. Only the generalization AUC will be considered. The statistical significances of AUC scores and differences therebetween were tested using DeLong's test [28].

The features were normalized to zero mean and unit variance. Feature selection was performed with a maximum of 20 features. The settings of the NN classifier k=1, kNN classifiers were CV: k=10, LOO: k=15 (the square root of total training samples) and for both ANN eps=0[20].

The average training and generalization AUC for each classifier is shown in Table II.

TABLE II
The median auc of the cross-validation (cv) and Leave-one-
out (LOO) feature selection evaluation for each Classifier.
Numbers in parenthesis are the standard deviation (std).

| | | Training | Generalisation |
|---|---|---|---|
| Number | Classifier | AUC | AUC |
| 1 | NN CV | 0.917 (0.036) | 0.592 (0.057) |
| 2 | NN LOO | 0.881 (0.033) | 0.611 (0.057) |
| 3 | wNN CV | 0.989 (0.015) | 0.692 (0.067) |
| 4 | wNN LOO | 0.965 (0.021) | 0.716 (0.072) |
| 5 | kNN k 10 CV | 0.964 (0.025) | 0.714 (0.066) |
| 6 | kNN k 14 LOO | 0.922 (0.017) | 0.750 (0.060) |
| 7 | wkNN k 10 CV | 0.988 (0.021) | 0.700 (0.072) |
| 8 | wkNN k 14 LOO | 0.940 (0.020) | 0.742 (0.065) |
| 9 | LDA CV | 0.989 (0.017) | 0.743 (0.064) |
| 10 | LDA LOO | 0.976 (0.013) | 0.771 (0.056) |
| 11 | QDA CV | 0.987 (0.020) | 0.648 (0.082) |
| 12 | QDA LOO | 0.978 (0.011) | 0.704 (0.071) |

Across all classifiers the median training AUC varies from 0.88 to 0.99 and the generalisation AUC from 0.59 to 0.77. Comparing CV and LOO show that LOO in general improves the generalization AUC but also worsen the training AUC. This means that the span between training and generalization AUC decreases as expected when increasing the training data by LOO.

When doing weighting of the nearest neighbours in the NN and kNN classifiers both training and generalisation AUC increase. For the wNN classifier the increase is extensive while minor for wkNN. This is probably because the weighting scheme includes more neighbours than usual classification and this makes a larger difference when solely relying on one neighbour compared to 14 neighbours in kNN.

LDA performs better than the other classifiers in spite of its linearity: both training and generalization AUC is higher for the LDA classifier. The training AUC of the QDA classifier is similar to the LDA but the generalization AUC is much lower. This indicates overfitting of the data when performing QDA. Including the single best feature resulted in training AUC between 0.666 and 0.812, and generalization AUC from 0.524 to 0.734. For all classifiers selecting a set of features yielded higher performance than choosing only a single feature.

The classification was done in the LOO LDA classifier scheme. The average generalization AUC for each biomarker and for the aggregate biomarkers is shown in Table III.

TABLE III
The Median AUC and the P-values for each biomarker. The P-value
for the biomarker vs the developed bone structure biomarker, and
last the P-value for the biomarker vs the aggregate marker.

| | | | P-value vs. | P-value vs. |
|---|---|---|---|---|
| | | | bone | aggregate |
| Biomarker | AUC | P-value | structure | all |
| CTX-II | 0.708 | 0.009 | 0.0001 | <0.0000 |
| Cartilage volume | 0.584 | 0.42 | 0.062 | 0.011 |
| Cartilage thickness | 0.597 | 0.41 | 0.062 | 0.008 |
| Cartilage smoothness | 0.792 | 0.009 | 0.58 | 0.33 |
| Cartilage curvature | 0.761 | 0.015 | 0.59 | 0.24 |
| Cartilage homogeneity | 0.673 | 0.096 | 0.26 | 0.041 |
| Bone structure | 0.771 | 0.0059 | — | 0.075 |
| Aggregate cart + CTX-II | 0.820 | 0.0037 | 0.45 | 0.53 |
| Aggregate all | 0.846 | 0.00059 | 0.075 | — |

The individual biomarkers spanned from AUC of 0.584, cartilage volume (p=0.42), to AUC of 0.792, cartilage smoothness (p=0.009). Both the biochemical marker, CTX-II, the cartilage markers smoothness and curvature, and the bone structure marker had an AUC above 0.70. In fact, the bone structure marker had the second highest AUC among the individual markers and the AUC score was significant (p=0.0059).

The bone structure marker AUC was significantly higher than CTX-II (p=0.0001), while not significant for the cartilage biomarkers. Combining all biomarkers except the developed bone structure marker to form an aggregate marker resulted in AUC 0.820 (p=0.0037). The aggregate biomarker including all biomarkers resulted in AUC of 0.846 (p=0.000059) which is the highest AUC, significantly higher than CTX-II, cartilage volume, cartilage thickness, and cartilage homogeneity.

A low sample size and a high dimensional feature space was the motive for performing bootstrapping in the CV and LOO schemes. It produced satisfying results. However, both CV and LOO resulted in 100 different bone structure sets, one for each evaluation. Further investigation of the features which describe the difference in OA status is difficult when dealing with 100 different feature sets. One could do equivalence class analysis of the feature sets to extract the overall features but it is a nontrivial task. Therefore, we proceeded with simplifying the evaluation scheme so that it results in a single set of features. The overall performance of this single feature set can then be included in an aggregate marker which is evaluated by its separation ability between the individual KL levels.

Determining the single best feature set was done by a single selection of features by SFFS using the LDA classifier.

However, to encompass the diversity of the data set, we included several different training and validation sets in the SFFS evaluation: For each evaluation of a feature set within SFFS, 100 different training and validation sets were used to assess the performance of the feature set. Each of the experiments resulted in an AUC measure. The overall performance of the feature set was calculated as the median AUC of the 100 experiments.

The resulting single best feature set was furthermore included into an aggregate marker. The aggregate marker consisted of the following markers: single best bone structure marker, cartilage volume, cartilage thickness, cartilage smoothness, cartilage curvature, cartilage homogeneity, and CTX-II as described above. The aggregate marker was evaluated by its capability of separating healthy and OA knees, but also OA in different stages. The statistical test used was Students t-test. Each feature was normalized to zero mean and unit variance based on the distribution of the training set. Feature selection was performed with a maximum of 20 features.

Selecting a single feature set resulted in training AUC 0.959 and generalization AUC 0.777 which was comparable to the LDA results of CV and LOO in Table II. The aggregate marker including the single best feature set resulted in AUC 0.926. The mean probability of being healthy for the healthy and OA groups, and for each KL level defined by the LDA classifier is shown in FIG. 2. The means of measurements are shown (with bars illustrating the standard error of the mean, SEM) for the groups healthy and OA and then, to the right of the dotted line, for each KL score. The levels where statistically significant separation is possible are marked by stars. There is a significant difference (p<0.0001) between the healthy group vs OA, shown left of the dotted line in the table. Furthermore, the difference between KL0 and KL1 (p<0.0001), KL1 and KL2 (p<0.0001), and KL2 and KL3-4 (p<0.01) were also significant.

The chosen features included N-jet filters, and both Eigen vectors and values for the Structure Tensor and Hessian Both the mean, standard deviation, and entropy scores at all three scales were included. In general, derivatives of order 1 and more in the y and z direction were chosen. This indicates a preference for features describing variation in the local orientation.

The results demonstrated the possibility of separating healthy and OA knees based on the trabecular bone structure. Trying several classifiers revealed that for this problem a linear classifier outperforms both the quadratic classifier and the non-linear classifiers NN and kNN. The properties of the data are believed to be the reason. We operated with high dimensional feature spaces when doing classification with a very limited data set. So, the feature space will be very sparse. When doing classification the simple, parametric LDA makes a general rule to separate data based on all available training samples. kNN, however, bases the classification on only the k nearest neighbours which will be distant from the test sample and thus different in features and possibly in class label. LDA follows the overall trend of the data while kNN bases the decision on a fraction of the data.

That LDA performs better than kNN indicates that the knees in each class, healthy or osteoarthritic, lie in a single cluster in feature space.

Selection of a set of appropriate features demonstrated better results than including the single best feature or the complete set of features. This suggests that feature selection is a necessary step in developing an imaging marker for bone structure.

Using the LOO scheme instead of the CV scheme doubled the size of the training data and the results showed an improvement for the generalization AUC. LOO is an appropriate choice when data is limited and overfitting is a risk. The diagnostic ability of the developed bone structure marker was comparable to the other biomarkers of OA. The bone structure marker AUC was significantly higher than several of the established MRI cartilage markers and the bio-chemical marker, showing the feasibility of developing MRI trabecular bone structure markers of OA.

The complexity and heterogeneity of OA makes it unlikely that a single marker will allow a comprehensive quantification. Therefore, aggregate markers based on measurements targeting different anatomical structures could potentially be superior. The results showed that the very different individual biomarkers each provided some diagnostic ability. However, the developed aggregate marker was superior, albeit not significantly, and demonstrated the potential for multi-modal markers including the biomarker of this invention.

Simplifying the evaluation scheme so that it resulted in a single set of features showed promising results. The generalization AUC of the bone structure features was comparable to LOO. The aggregate marker including the single feature set showed significant separation between the healthy and the OA group, and between the individual KL levels, so that that the developed aggregate biomarker shows utility as a diagnostic as well as an efficacy marker for OA treatment.

The single feature set included features related to the local orientation within the trabecular bone. This suggests that the bone structure which differs between the healthy and the diseased is indeed related to the trabecular architecture, as expected. Furthermore, preliminary analysis of the selected features suggests that under machine analysis the OA bone texture appears inhomogeneous in low-field MRI as compared to the healthy bone.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

[1] G. Blumenkrantz, C. T. Lindsey, T. C. Dunn, H. Jin, M. D. Ries, T. M. Link, L. S. Steinbach, and S. Majumdar, “A pilot, two-year longitudinal study of the interrelationship between trabecular bone and articular cartilage in the osteoarthritic knee,” Osteoarthritis and Cartilage, vol. 12, no. 12, pp. 997-1005, December 2004.
[2] J. H. Kellgren and J. S. Lawrence, “Radiological assessment of osteo-arthrosis,” Annals of the Rheumatic Diseases, vol. 16, no. 4, pp. 494-502, December 1957.
[3] R. W. Moskowitz, R. D. Altman, M. C. Hochberg, J. A. Buckwalter, and V. M. Goldberg, Osteoarthritis: Diagnosis and Medical/Surgical management, 4th ed. Wolters Kluver Health, 2007.
[4] J. Folkesson, E. B. Dam, 0. F. Olsen, M. A. Karsdal, P. C. Pettersen, and C. Christiansen, “Automatic quantification of local and global articular cartilage surface curvature: Biomarkers for osteoarthritis?” Magnetic Resonance in Medicine, vol. 59, no. 6, pp. 1340-1346, 2008.
[5] E. B. Dam, J. Folkesson, P. C. Pettersen, and C. Christiansen, “Automatic morphometric cartilage quantification in the medial tibial plateau from MRI for osteoarthritis grading,” Osteoarthritis and Cartilage, vol. 15, no. 7, pp. 808-818, 2007.
[6] F. Eckstein, D. Burstein, and T. M. Link, “Quantitative mri of cartilage and bone: degenerative changes in osteoarthritis,” NMR in Biomedicine, vol. 19, pp. 822-854, 2006.
[7] C. G. Peterfy, A. Guermazi, S. Zaim, P. F. Tirman, Y. Miaux, D. White, M. Kothari, Y. Lu, K. Fye, S. Zhao, and H. K. Genant, “Whole-organ magnetic resonance imaging score (worms) of the knee in osteoarthritis,” Osteoarthritis and Cartilage, vol. 12, no. 3, pp. 177-190, 2004.
[8] F. W. Roemer and A. Guermazi, “MR imaging-based semiquantitative assessment in osteoarthritis,” Radiologic Clinics of North America, vol. 47, no. 4, pp. 633-654, July 2009.
[9] P. R. Kornaat, R. Y. T. Ceulemans, H. M. Kroon, N. Riyazi, M. Klop-penburg, W. O. Carter, T. G. Woodworth, and J. L. Bloem, “Knee osteoarthritis scoring system (koss) inter-observer and intra-observer reproducibility of a compartment-based scoring system,” Skeletal Radiology, vol. 34, pp. 95-102, February 2005.
[10] A. M. Parfitt, M. K. Drezner, F. H. Glorieux, J. A. Kanis, H. Malluche, P. J. Meunier, S. M. Ott, and R. R. Recker, “Bone histomorphometry: standardization of nomenclature, symbols and units,” Journal of Bone Mineral Research, vol. 2, no. 6, pp. 595-610, 1987.
[11] E. A. Messent, J. C. Buckland-Wright, and G. M. Blake, “Fractal analysis of trabecular bone in knee osteoarthritis (oa) is a more sensitive marker of disease status than bone mineral density (bmd),” Calcified Tissue International, vol. 76, no. 6, pp. 419-425, 2005.
[12] J. C. Buckland-Wright, J. A. Lynch, and D. G. Macfarlane, “Fractal signature analysis measures cancellous bone organisation in macrora-diographs of patients with knee osteoarthritis,” Annals of the Rheumatic Diseases, vol. 55, no. 10, pp. 749-755, 1996.
[13] E. A. Messent, R. J. Ward, C. J. Tonkin, and C. Buckland-Wright, “Differences in trabecular structure between knees with and without osteoarthritis quantified by macro and standard radiography, respectively,” Osteoarthritis and Cartilage, vol. 14, no. 12, pp. 1302-1305, 2006.
[14] V. Patel, A. S. Issever, A. Burghardt, A. Laib, M. Ries, and S. Majumdar, “Microct evaluation of normal and osteoarthritic bone structure in human knee specimens,” Journal of Orthopaedic Research, vol. 21, no. 1, pp. 6-13, 2006.
[15] F. Eckstein, A. Guermazi, and F. W. Roemer, “Quantitative MR imaging of cartilage and trabecular bone in osteoarthritis,” Radiologic Clinics of North America, vol. 47, no. 4, pp. 655-673, July 2009.
[16] J. Folkesson, E. B. Dam, O. F. Olsen, P. C. Pettersen, and C. Chris-tiansen, “Segmenting articular cartilage automatically using a voxel clas-sification approach,” IEEE Transactions on Medical Imaging, vol. 26, pp. 106-115, 2007.
[17] E. B. Dam, J. Folkesson, P. C. Pettersen, and C. Christiansen, “Automatic cartilage thickness quantification using a statistical shape model,” in MICCAI Joint Disease Workshop, 2006.
[18] L. M. J. Florack, B. M. t. Haar Romeny, J. J. Koenderink, and M. A. Viergever, “The Gaussian scale-space paradigm and the multiscale local jet,” International Journal of Computer Vision, vol. 18, no. 1, pp. 61-75, April 1996.
[19] K. Chernoff and M. Nielsen, “Weighting of the k-Nearest-Neighbors,” International Conference of Pattern Recognition (ICPR), 2010.
[20] S. Arya, D. M. Mount, N. S. Netanyahu, R. Silverman, and A. Y. Wu, “An optimal algorithm for approximate nearest neighbor searching,” Journal of the ACM, vol. 45, no. 6, pp. 891-923, November 1998.
[21] P. Somol, P. Pudil, J. Novovic\check{}ova', and P. Paclk, “Adaptive floating search methods in feature selection,” Pattern Recognition Letters, vol. 20, no. 11-13, pp. 1157-1163, 1999.
[22] I. Guyon, S. Gunn, M. Nikravesh, and L. A. Zadeh, Feature Extrac-tion: Foundations and Applications, ser. Studies in Fuzziness and Soft Computing. Springer Berlin/Heidelberg, 2006, vol. 207.
[23] P. Pudil, J. Novovic\check{}ova', and J. Kittler, “Floating search methods in feature selection,” Pattern Recognition Letters, vol. 15, no. 11, pp. 1119-1125, 1994.
[24] A. K. Jain and D. Zongker, “Feature selection: Evaluation, application, and small sample performance,” IEEE Transactions on Pattern Analysis and Machine Intelligence, vol. 19, no. 2, pp. 153-158, 1997.
[25] M. Sonka and J. M. Fitzpatrick, Handbook of Medical Imaging—Medical Image Processing and Analysis. SPIE, 2000, vol. 2.
[26] E. B. Dam, M. Loog, C. Christiansen, I. Byrjalsen, J. Folkesson, M. Nielsen, A. Qazi, P. C. Pettersen, P. Garnero, and M. A. Karsdal, “Identification of progressors in osteoarthritis by combining biochem-ical and MRI-based markers,” Arthritis Research & Therapy, vol. 11, no. 4, p. R115, 2009, see related editorial by Williams, http://arthritis-research.com/content/11/5/130.
[27] A. A. Qazi, J. Folkesson, P. C. Pettersen, M. A. Karsdal, C. Christiansen, and E. Dam, “Separation of healthy and early osteoarthritis by automatic quantification of cartilage homogeneity,” OsteoArthritis and Cartilage, vol. 15, no. 10, pp. 1199-1206, 2007.
[28] E. R. DeLong, D. M. DeLong, and D. L. Clarke-Pearson, “Comparing the areas under two or more correlated receiver operating characteristic curves: a nonparametric approach,” Biometrics, vol. 44, no. 3, pp. 837-845, 1988.

1. A method for computer based analysis of a low field MR image including a trabecular region of bone for extracting from said image diagnostic information, said method comprising applying to said image a trained statistical classifier which has been trained on a training set of low field MRI images containing said trabecular region of bone each of which images has been labelled according to the severity of a trabecular bone altering disease suffered by a person from whom the image derived, wherein in said training of the classifier, for each image in the training set a region of interest (ROI) was defined, and textural information relating to the intensities of voxels within the ROI was obtained, and combinations of features of said textural information were found which suitably classify said training set images according to said labelling, and wherein, in applying said trained statistical classifier to said image, in a computer the region of interest (ROI) is found in said image, and textural information relating to the intensities of voxels within the ROI of the kind used in training the classifier is obtained, and features of said textural information for the voxels within the ROI of the image are combined as learnt in the training of the classifier to estimate a level of said trabecular bone altering disease or propensity to develop said bone altering disease or a level thereof associated with said image.

2. A method as claimed in claim 1, wherein said training set images were labelled according to the severity of trabecular bone altering disease suffered by the person to whom the image relates at the time of taking the image.

3. A method as claimed in claim 1, wherein said training set images were labelled according to the severity of trabecular bone altering disease suffered by the person to whom the image relates at a time subsequent to the time of taking the image and wherein the image was taken at a time when the said person did not suffer from the disease or suffered from the disease to a lesser extent.

4. A method as claimed in claim 1, wherein said trabecular bone altering disease is arthritis.

5. A method as claimed in claim 1, wherein the trabecular bone altering disease is osteoporosis or Paget's disease of the bone.

6. A method as claimed in claim 1, wherein the training set images and the image to be analyzed are acquired using an MRI apparatus having a field strength of not more than 0.5 T.

7. A method as claimed in claim 1, wherein said textural information includes textural information obtained by applying to the image one or more of the following filters: N-jet, Structure Tensor, Hessian, Gradient, and Gradient Magnitude and deriving for each filtered image one or more of the mean, standard deviation and Shannon entropy.

8. A method as claimed in claim 7, wherein said filters are applied at multiple scales.

9. A method as claimed in claim 7, wherein said textural information further includes textural information obtained by deriving from the unfiltered image one or more of the mean, standard deviation and Shannon entropy.

10. A method as claimed in claim 7, wherein said textural information includes textural information obtained by applying to the image the N-jet, Structure Tensor and Hessian filters at multiple scales and deriving for each filtered image one or more of the mean, standard deviation and Shannon entropy.

11. A method as claimed in claim 1, wherein said estimation is combined with one or more other biomarkers estimating the present or future extent of said trabecular bone altering disease in the person from whom the image derives, so as to form a composite biomarker.

12. A method as claimed in claim 11, wherein said one or more other biomarkers is or are selected from the group consisting of a biochemical cartilage breakdown product measure, cartilage volume, cartilage thickness, cartilage smoothness, cartilage curvature and cartilage homogeneity.

13. A method as claimed in claim 1, wherein a second image obtained from the same person at an earlier or later time is also similarly analyzed and the results of the analysis for the two images are compared.

14. A method for the development of a statistical classifier for computerised classification of a low field MR image including a trabecular region of bone for extracting from said image diagnostic information, said method comprising training a statistical classifier on a training set of low field MR images containing said trabecular region of bone each of which images has been labelled according to the severity of a trabecular bone altering disease suffered by a person from whom the image derived, wherein in said training of the classifier, for each image in the training set a region of interest (ROI) is defined, and textural information relating to the intensities of voxels within the ROI are obtained, and combinations of features of said textural information are found which suitably classify said training set images according to said labelling.
